(19) United States
(12) Patent Application Publication
(10) Pub. No.: US 2003/0130336 A1
(43) Pub. Date: Jul. 10, 2003

(54) Method of determining the activity of a central nervous system drug on striatal dopamine transporter populations

(75) Inventors: Peter J. Snyder, West Hartford, CT (US); Robert B. Innis, Rockville, MD (US); John P. Seibyl, Branford, CT (US); Akira Kugaya, New Haven, CT (US); Stephen A. Williams, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/334,239

(22) Filed: Dec. 31, 2002

Related U.S. Application Data
(60) Provisional application No. 60/345,799, filed on Jan. 2, 2002.

(51) Int. Cl. A61K 51/00; A61K 31/405

(52) U.S. Cl. 514/419; 424/1.11

(57) ABSTRACT

This invention relates to a method of predicting the activity of a CNS drug on striatal dopamine transporter populations. The method uses SPECT brain imaging to predict the activity of antidepressants or anxiolytic agents on striatal dopamine transporter populations. The method uses the increase in dopamine transporter populations as a marker for predicting sexual dysfunction side effects, as well as to determine proper dosing for antidepressants or anxiolytics agents in order to avoid the sexual dysfunction side effects.

### BACKGROUND

This invention relates to a method of determining the activity of a pharmaceutical drug, preferably a drug affecting the Central Nervous System (CNS), and in particular an antidepressant or anxiolytic agent on striatal dopamine transporter populations. Sexual dysfunction side-effects of selective serotonin reuptake inhibitors (SSRI), for treatment of depression or anxiety disorders (defined as unwanted pharmacotherapy-related changes in any of the four key stages of the human sexual response cycle [i.e., drive, arousal, orgasm & resolution]), is fairly commonly reported. Rosen R C, Lane, R M, Menza, M (1999). Effects of SSRIs on sexual function: A critical review. J. Clin Psychopharmacol, 19, 67-85. Among the sexual side effects most commonly associated with SSRI use are delayed ejaculation and absent or delayed orgasm. Sexual desire and arousal difficulties are also frequently reported. The effects of SSRIs on sexual dysfunction appear to be dose-related and may vary within this drug class depending on the pharmacologic characteristics of specific drugs (e.g., effect on serotonin and dopamine reuptake mechanisms, induction of prolactin release, anticholinergic effects).

Some studies have shown a greater incidence of sexual dysfunction in patients receiving paroxetine, compared with those receiving sertraline, fluoxetine, and fluvoxamine. This finding may be related to the relatively high potency of paroxetine for serotonin reuptake inhibition together with supposedly little or no effect on dopamine reuptake (see review by Rosen et al., 1999). Nonetheless, dopamine transmission has consistently been shown to be involved in sexual arousal and numerous uncontrolled studies have supported the rationale for use of dopamine agonists for treatment of SSRI-induced ejaculatory dysfunction (see review by Rosen et al., 1999). However, little or no information is currently available regarding the direct effect that SSRIs may exert on the dopaminergic system itself (e.g., the dopamine transporter system in the striatal nuclei).

### SUMMARY OF THE INVENTION

This invention relates to a method of determining the activity of a CNS drug, in particular an antidepressant or anxiolytic agent, on striatal dopamine transporter population. The method uses Single Photon Emission Computed Tomography (SPECT) brain imaging, with the [¹²³I] β-CIT radioligand, to predict the activity of antidepressant or anxiolytic agents on striatal dopamine transporter (DAT) populations. The SPECT scan is used to image the change in dopamine transporter (DAT) and serotonin transporter (SERT) populations caused by taking the antidepressant or anxiolytic agents. The central finding supporting this invention is the observation that steady-state administration of an antidepressant or anxiolytic agent leads to an increase in the striatal DAT population. Unwanted direct effects of SSRIs on this system may lead to the very side effects that are treated by use of concomitant therapy with dopamine agonists. This increase in striatal DAT is directly observed at steady-state dosing with a “pure” SSRI, and is an SSRI class effect. Examples of “pure” SSRI antidepressants or anxiolytic agents are citalopram, fluoxetine hydrochloride, sertraline, fluvoxamine maleate, and paroxetine hydrochloride. The method uses the increase in the DAT population to predict sexual dysfunction side effects with an SSRI antidepressant or anxiolytic agent. This method leads to the observation of a relative increase in the ratio of specific-to-nonspecific binding of the labeled ligand, eg [¹²³I] β-CIT, in the striatum (V3” ratio) of about 1.0 to about 2.0 units at steady-state dosing on an SSRI agent. This novel finding leads to a new method for determining proper dosage for SSRI agents, to avoid associated sexual dysfunction side effects. This novel finding also leads to a method for screening potential drug compounds to determine their affects on DAT populations.

### BRIEF DESCRIPTION OF THE DRAWINGS

FIG. 1 shows the ratio (referred to as V3”) of specific to non-specific binding of the radioligand to the dopamine transporters (DAT) in the striatal nuclei, for each of two subjects groups, and at each of 3 SPECT scan timepoints (baseline, Day 8, and Day 16).

FIG. 2 shows the ratio (referred to as V3”) of specific to non-specific binding of the radioligand to the serotonin transporters (SERT) in the midbrain and diencephalon (striatal nuclei), for each of two subjects groups, and at each of 3 SPECT scan timepoints (baseline, Day 8, and Day 16).

### DETAILED DESCRIPTION OF THE INVENTION

This invention provides evidence to support the use of a specific SPECT radioligand for a novel purpose. That is, to image striatal DAT changes at steady-state on an SSRI antidepressant or anxiolytic, as a marker of dopamine-related side effects, and in particular, to guide dose selection for novel compounds to avoid sexual dysfunction side effects.

The invention seeks, in one embodiment, to determine the effect of a “pure” SSRI (citalopram etc.) on the DAT in the striatum, in order to help explain why SSRI medications lead to a diminishment in dopaminergic activity and sexual dysfunction side effects. A “pure” SSRI is defined as a psychotherapeutic agent that has been designed to principally act by inhibiting the reuptake of serotonin from the synaptic cleft. These medications principally affect the serotonergic system and are not intended to affect multiple neurotransmitter systems. The invention shows, in one embodiment, how an SSRI might lead to an increase in DAT striatal populations, thereby leading to more efficacious removal of the neurotransmitter from the synapse (possibly resulting in a subclinical hypodopaminergic state), and this can then be used as a marker for predicting sexual dysfunction side effects in novel compounds. By determining the percent occupancy of the radioligand to the DAT with steady-state dosing of an SSRI, in another embodiment of the invention, this can be used as a “benchmark” to both predict the risk of these side effects and to modify dosing in order to reduce this risk.

Measurement of the binding of both citalopram and bupropion HCI to the dopamine and serotonin transporters was made by evaluating the extent of displacement of [¹²³I] 2β-carbomethoxy-3β-(4-iodophenyltropane), also known as [¹²³I] β-CIT, off of these same binding sites. This SPECT radioligand has a high affinity for both dopamine and serotonin transporters, and has been used as a SPECT imaging probe in human and nonhuman primates. Our aim was to determine how effectively the two study drugs (citalopram and bupropion HCI), both independently and in combination, displace this radioligand off of both the serotonin transporters (in the midbrain region) and dopamine transporters (in the striatal nuclei).

Group 1 (sample size of approximately 8 subjects) received escalating doses of bupropion hydrochloride (Wellbutrin©, SR formulation), at two dosing levels, over a 16-day period. These subjects completed SPECT imaging at baseline, at the end of the first 8 (±1) days, and then again at the end of the second 8-day period (±1 day).

Group 2 (sample size of approximately 8 subjects) received a single dosing level of citalopram (Celexa©) over a single 8-day period (±1 day). Following this drug treatment period, these subjects continued for a second 8-day period on the same dose of citalopram, with the addition of a low dose of bupropion HCI administered concomitantly during study days 9 to 16 (±1 day). These subjects completed SPECT imaging at baseline, at the end of the first 8 (±1) days, and then again at the end of the second 8-day period (±1 day).

Group 1, Days 1-8 (N=8):

Bupropion hydrochloride, sustained-release formulation (Wellbutrin-SR©) at a daily dose of 100 mg po qAM, with breakfast.

Group 1, Days 9-16 (N=8):

Bupropion hydrochloride, sustained-release formulation (Wellbutrin-SR©) at a daily dose of 100 mg po qBID, with breakfast and dinner.

Group 2, Days 1-8 (N=8):

Citalopram (Celexa©) at a daily dose of 20 mg po qBID, with breakfast and dinner.

Group 2, Days 9-16 (N=8):

Citalopram (Celexa©) at a daily dose of 20 mg po qBID, with breakfast and dinner; and bupropion hydrochloride, sustained-release formulation (Wellbutrin-SR©) at a daily dose of 100 mg po qAM, with breakfast.

A total of 17 healthy volunteer subjects, meeting the inclusion and exclusion criteria provided below, were included in this study. There was final total of 8 subjects in group 1, and 9 subjects in group 2, as described below. Determination of sample sizing accounted for the possibility of early withdrawal from participation by up to two subjects receiving Wellbutrin-SR© and/or Celexa© secondary to uncomfortable medication side effects (Gitlin et al. 2000; Wellbutrin-SR© and Celexa© product label data).

1. Adults aged 18-55 years

2. Males and females: all females were of non-child bearing potential (e.g. postmenopausal, infertile induced by surgically-approved methods) or using appropriate oral contraception.

3. Written informed consent was given in the study.

4. Subjects weighed no more than 210 pounds, (91 kg) and no less than 120 pounds (54 kg) and were within 15% of their weight range for age, gender, height and frame as established in the “1983 Metropolitan Life Insurance Height and Weight Tables” (Appendix E).

5. Subjects were willing and able to both take all study medications as directed, and to attend as out-patients as often as required to complete all phases of the study.

All subjects did complete a single MRI scan, scheduled at the subject's convenience (see Table 1), at any point following their screening visit and prior to their last SPECT scan. This scan was completed at the MR facility housed within the same building as the SPECT facility (40 Temple Street, New Haven, Conn.).

The MR acquisition protocol was as follows: Spoiled Volume Gradient Recall Acquisition in Steady State (SPGR): TR 25, TE 5, flip angle 45°, FOV 24, 3 mm slice thickness, no gap, 256×192 matrix, 2 nex). The purpose of the MR scan was to direct the region of interest placement on the lower resolution SPECT study. SPECT and MRI images were coregistered using software which the study site investigators have developed in collaboration with Eric Miller and Robert Riker of Coritechs, Inc. (New Haven, Conn.).

The specific study design, that has led to this invention, is both described below and depicted in Table 1.

TABLE 1

| | | | | Drug | | | Drug | Day 16 |
|---|---|---|---|---|---|---|---|---|
| | | Day 0 | Day 0 | Rx. | Day 8 | Day 8 | Rx. | SPECT: |
| | | SPECT: | SPECT: | Days | SPECT: | SPECT: | Days | Groups |
| Event/Item | Screening | Group 1 | Group 2 | 1-8 | Group 1 | Group 2 | 9-16 | 1 & 2 |
| Full Physical | X | | | | | | | |
| ECG | X | | | | | | | |
| Laboratory | X | | | | | | | |
| Safety Tests | | | | | | | | |
| Randomization | X | | | | | | | |
| Citalopram | | | | Group | | | Group | |
| 20 mg po BID | | | | 2 | | | 2 | |
| Bupropion HCl | | | | Group | | | Group | |
| 100 mg po qAM | | | | 1 | | | 2 | |
| Bupropion HCl | | | | N/A | | | Group | |
| 100 mg po qBID | | | | | | | 1 | |
| Blood sampling | | | | X | X (for | | X | |
| for plasma | | | | | | possible | | |
| concentrations | | | | | | assay of | | |
| of bupropion HCl | | | | | | Celexa © ) | | |
| & metabolites | | | | | | | | |
| Urine/pregnancy | X | X | X | | X | X | X | |
| Test | | | | | | | | |
| MR Scan | One Magnetic Resonance Scan Anytime Following Screening Exam and Prior to | | | | | | | |
| | Final SPECT Scan | | | | | | | |
| Radioligand | | X | X | | X | X | | X |
| Administration | | | | | | | | |
| 23 hr prior to | | | | | | | | |
| scan | | | | | | | | |

Pre-dosing With Spect Radioligand (day-1, 17 subjects)

Approximately 2-3 weeks following the screening visit, subjects were pre-treated with the radioligand, in preparation for the first SPECT scan at approximately 23 hours following injection (laboratory safety results must be reviewed prior to SPECT scan 1). Subjects arrived at the SPECT facility for their scheduled appointments (this was not a pharmacokinetics study, and there were no restrictions on food intake). For each SPECT scan in this study, all subjects were pretreated with stable iodine (10 drops of a saturated solution of potassium iodide) on the morning of the day of dosing on the radioligand, to reduce thyroid uptake of ¹²³I. Each subject then received an intravenous injection of [¹²³I]β-CIT, at a dosage of 6 mCi (single bolus injection), approximately 23 hours prior to their scheduled SPECT scan. Each subject completed a baseline SPECT scan, and each follow-up scan (see below) conformed to these same procedures.

SPECT Scan 1 (day 0, 17 subjects)

A single SPECT scan (˜1 hr in duration) was completed at approximately 23 hours post-dosing with [¹²³I]β-CIT. Immediately prior to each SPECT scan, four fiducial markers filled with 4-5 μCi of [99mTc]NaTcO4 was attached to both sides of the subject's head at the level of the canthomeatal line prior to imaging to facilitate post hoc computer reorientation of transaxial images.

During SPECT imaging, venous blood samples (10 ml) were obtained via “butterfly” needle in order to measure plasma [¹²³I]β-CIT.

Projection data was acquired on the PICKER PRISM 3000 camera fitted with high resolution fanbeam collimators (acquisitions obtained for 30 min, 128×128 matrix) into a 20% symmetric window centered on 159 keV at 24+2 hours post injection. Raw projection data was prefiltered with a Butterworth filter (power=10, cut-off=0.26 cm) and SPECT images were generated using a ramp filter. These filters were optimized for quantitative assessment. In addition, a transmission scan was acquired using a 15 mCi ⁵⁷Co line source affixed to the camera gantry. Simultaneous emission and transmission data was acquired in one head, while the other two heads detected only emission data and were used to subtract the emission photons from the transmission scan. The transmission scan provided an anatomical reference for accurate attenuation correction.

Drug Treatment—Block 1 (days 1-8, 17 subjects)

Following the baseline SPECT scan on Day 0, all subjects randomized to group 1 were provided with an 8 day supply of bupriopion hydrochloride (sustained-release formulation) at a dosage of 100 mg po qAM. All subjects in group 1 were required to take this medication as directed, once per day at breakfast time, for 8 days.

Following the baseline SPECT scan on Day 0, all subjects randomized to group 2 were provided with an 8 day supply of citalopram at a dosage of 20 mg po BID. All subjects in group 2 were required to take this medication as directed, twice per day at breakfast and dinner times, for 8 days. This dosing schedule was based on that reported by Pirker W, Asenbaum S, Kasper S, et al. (1995) β-CIT SPECT demonstrates blockade of 5HT-uptake sites by citalopram in the human brain in vivo. J. Neural Transmission [Gen Sect], 100, 247-256.

Pre-dosing With Spect Radioligand (day 7, 17 subjects)

On day 7, all subjects were injected with the radioligand, in preparation for the 2^{nd} SPECT scan at approximately 23 hours following pre-treatment. All subjects were pretreated with stable iodine (10 drops of a saturated solution of potassium iodide) on the morning of the day of dosing on the radioligand, to reduce thyroid uptake of ¹²³I. Each subject then received an intravenous injection of [¹²³I]β-CIT, at a dosage of 6 mCi (single bolus injection), approximately 23 hours prior to their 2^{nd} scheduled SPECT scan on day 8.

Spect Scan 2 (day 8, 17 subjects)

To measure circulating plasma concentrations of bupropion HCI and its principal metabolites, for subjects in group 1, blood sufficient to provide a minimum of 2 ml plasma was collected in tubes containing EDTA. Samples were centrifuged at approximately 4° C. and the plasma was stored in appropriately labeled, screw-capped polypropylene tubes at −20° C. within 1 hour of collection. Samples from each individual subject was stored as a package for that subject, and at the end of the study these samples were shipped to an independent laboratory for analysis. As a reliable blood assay for circulating levels of citalopram, we also collected from group 2 enough blood to provide a minimum of 2 ml plasma, in tubes containing EDTA. These samples were handled and stored as described above.

Following this, a single SPECT scan (˜1 hr in duration) was completed at approximately 23 hours post-dosing with [¹²³]β-CIT. The specific methods for this procedure were described above, for the baseline SPECT scan (day 0).

Drug Treatment—Block 2 (days 9-17 subjects)

Following the 2^{nd} SPECT scan on Day 8, all subjects randomized to group 1 was provided with an 8 day supply of bupriopion hydrochloride (sustained-release formulation) at a dosage of 100 mg po BID. All subjects in group 1 were required to take this medication as directed, twice per day at breakfast and dinner times, for 8 days (beginning the following morning, on day 9).

Following the 2^{nd} SPECT scan on Day 8, all 6 subjects randomized to group 2 were provided with a second 8 day supply of citalopram at a dosage of 20 mg po BID, as well as an 8 day supply of bupriopion HCI (sustained-release formulation) at a dosage of 100 mg qAM. These subjects were asked to take both medications, as directed, concomitantly for approximately 8 days (study days 9-16, ±1).

Pre-Doding with SPECT Radioligand (day 15, 17 subjects)

On day 15, all subjects randomized to group 1 were injected with the radioligand, in preparation for the 3^{rd} SPECT scan at approximately 23 hours following pre-treatment. All subjects were pretreated with stable iodine (10 drops of a saturated solution of potassium iodide) on the morning of the day of dosing on the radioligand, to reduce thyroid uptake of ¹²³I. Each subject received an intravenous injection of [¹²³I]β-CIT, at a dosage of 6 mCi (single bolus injection), approximately 23 hours prior to their 3^{rd} scheduled SPECT scan on day 16.

SPECT Scan 3 (day 16, 17 subjects)

To measure circulating plasma concentrations of bupropion HCI and its principal metabolites, for all subjects in both groups 1 and 2, blood sufficient to provide a minimum of 2 ml plasma was collected in tubes containing EDTA. Samples were centrifuged at approximately 4° C. and the plasma was stored in appropriately labeled, screw-capped polypropylene tubes at −20° C. within 1 hour of collection. Samples from each individual subject were stored as a package for that subject, and at the end of the study these samples were shipped to an independent laboratory for analysis.

A single SPECT scan (˜1 hr in duration) was completed at approximately 23 hours post-dosing with [¹²³I]β-CIT. The specific methods for this procedure were described above, for the baseline SPECT scan (day 0).

Following completion of this 3^{rd} SPECT scan on day 16, all subjects randomized to groups 1 and 2 were withdrawn from their study medications. These subjects had also completed their MRI scan by this time, and following this 3^{rd} SPECT scan they had successfully completed their participation in this study.

A quantitative analysis of striatal DAT binding was computed for all subjects (in each group) and for every set of reconstructed SPECT scan data acquired (see above). Blinded image processing and determination of specific:non-displaceable striatal uptake ratio involved the following four steps: a. Attenuation correction: Attenuation ellipses were fit to the transaxial data for application of a Chang zero order (homogeneous) correction. Twenty-four hour uptake of [¹²³I] β-CIT was low in cortical areas and placement of the ellipses was difficult. The linear attenuation coefficient (p) was empirically based on: a. [¹²³I] distributed source phantom. b. Reorientation of axial slices along the canthomeatal line: Slice reorientation was performed to align images parallel to the canthomeatal plane. This is a user-operated, iterative process. c. Summation of striatal slices: On the reoriented image filet the striatal slice with the most intense uptake is determined by thresholding the color scale (index slice). The two slices above this slice, the index slice, and one slice below are summed. d. Placement of region of interest (ROI) template and extraction of count density data. The standard template contains right and left caudate, right and left putamen, and the posterior portions of both occipital lobes. These regions were developed on the basis of MRI scans obtained in the same healthy subjects and co-registered with [¹²³I]β-CIT images. Using strict criteria the nuclear medicine physician drew regions of interest and calculated the outcome measures. Specific-to-nondisplaceable activity ratios were determined by subtracting occipital densities (nondisplaceable uptake) from total caudate or putamen count densities (specific+nondisplaceable uptake) from four summed slices and dividing by occipital counts. To enable additional review of the quantitative analysis, details of attenuation correction ellipse, data reorientation angles and the summed slices was noted. The data collected at each stage of the analysis including the raw projection data, reconstructed data, attenuation corrected data, reoriented data, summed striatal slice data, and ROI file data was stored.

High specific activity [¹²³I] β-CIT was prepared from the corresponding trimethylstannyl precursor, supplied by Research Biochemicals International, Natick, Mass., and high radionuclidic purity [¹²³I]Nal (Nordion International, Ltd., Vancouver, B. C., Canada) as previously described: (Seibyl, et al., 1995b). Seibyl, J P, Marek K, Sheff K, Baldwin R M, et al. (1997). Test/retest reproducibility of Iodine-123-β-CIT SPECT brain measurement if dopamine transporters in Parkinson's patients. J. Nucl Med, 38, 1453-1459. In previous studies radiochemical purity was 98+1% as measured by high-performance liquid chromatography (HPLC). Specific activity was >5000 Ci/mmol.

The primary outcome measure was a ratio of specific (i.e., ROI—occipital, where ROI refers to striatal activity for measures of the DA transporter and midbrain activity for the 5 HT transporter) to non-specific (i.e., occipital) binding. This specific to non-specific volume of distribution, also referred to as V3”, was proportional to transporter number (i.e., Bmax) under equilibrium conditions given assumptions of invariant transporter affinity and non-specific binding. Marek K, Seibyl J, et al. (1996). [¹²³]CIT SPECT imaging demonstrates bilateral loss of dopamine transporters in hemi-Parkinson's disease. Neurology, 46, 231-237. The goal for statistical analyses of the data collected was to simply determine the change in DAT and SERT receptor occupancy from baseline (both groups) to the steady-state dosing conditions on buproprion hydrochloride at 2 dosing levels (Group 1) or citalopram (group 2). No further division of subjects into additional subgroups was attempted, over that described within this protocol (see above).

Statistical Analysis

Data was evaluated using repeated-measures analysis of variance (RMANOVA) with within-subject factor of time (scan 1, 2 and 3) for each group. The RMANOVAs were tested for lack of sphericity and Huynh-Feldt adjustments were made to the degree of freedom to reduce type I error. A significant main effect by RMANOVA was followed by paired t-test with Bonferroni correction. All the analysis was done by SPSS 10.0.0.

Results

1) DAT binding in striatum

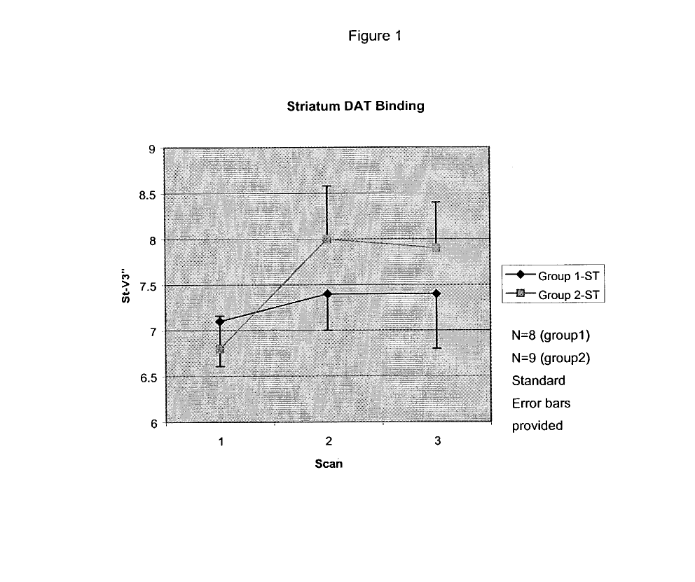

In Group 1, there was no significant main effect in the striatum DAT binding. In Group 2, a significant main effect was found. The DAT binding was increased after 8 days citalopram (p<0.012 Bonferroni correction, see Table 2) while no change was observed between scan 2 and 3 (see FIG. 1). The scan 3 DAT binding was still significantly higher than scan 1.

TABLE 2

| | | Scan 1 | Scan 2 | Scan 3 | p* |
|---|---|---|---|---|---|
| St-V3″ | Group 1 | 7.09 ± 1.38 | 7.42 ± 1.13 | 7.37 ± 1.69 | 0.38 |
| (Mean ± SD) | Group 2 | 6.83 ± 1.07 | 7.99 ± 1.73^{a} | 7.88 ± 1.51^{b} | 0.005 |

*RMANOVA (main effect)
^{a}p = 0.012 compared with Scan 1 (paired t-test)
^{b}p = 0.006 compared with Scan 1 (paired t-test)

2) SERT binding in midbrain and diencephalon

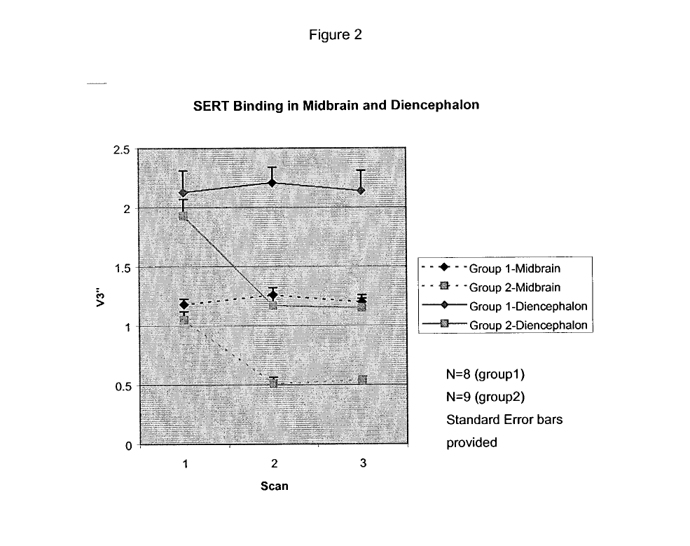

In Group 1, there was no significant main effect in the midbrain and diencephalon SERT through the study. In Group 2, significant decrease in the midbrain and diencephalon SERT binding between Scan 1 and the other 2 scans was found as shown in Table 3 and FIG. 2.

TABLE 3

| | | Scan 1 | Scan 2 | Scan 3 | p* |
|---|---|---|---|---|---|
| Mb-V3″ | Group 1 | 1.18 ± 0.13 | 1.26 ± 0.18 | 1.20 ± 0.17 | 0.10 |
| (Mean ± | Group 2 | 1.05 ± 0.21 | 0.51 ± 0.16^{a} | 0.54 ± 0.09 | <0.001 |
| SD) | | | | | |
| Die-V3″ | Group 1 | 2.13 ± 0.51 | 2.21 ± 0.36 | 2.14 ± 0.49 | 0.70 |
| (Mean ± | Group 2 | 1.93 ± 0.41 | 1.17 ± 0.29^{b} | 1.15 ± 0.24 | <0.001 |
| SD) | | | | | |

*RMANOVA (main effect)
^{a}p < 0.001 compared with Scan 1 (paired t-test)
^{b}p < 0.001 compared with Scan 1 (paired t-test)

The principle finding is that citalopram showed significant changes in the striatal DAT, as well as the expected decrease in both midbrain and diencephalic SERT binding, after 8 days. Administration of bupropion itself did not lead to any significant changes.

Bupropion is known to block the DAT in vivo, which may subsequently upregulate the transporter. There are two explanations for this: One possible explanation for unchanged DAT binding in the present study is that bupropion caused upregulation of the DAT, and [¹²³I]β-CIT binding remained unchanged because bupropion blocked (occupied) DAT in similar degree to the upregulation. Alternatively the dose of bupropion was not enough to change the DAT binding. A previous report that failed to show decrease in plasma prolactin by therapeutic dose (200 mg) of bupropion may support this possibility. Laakmann G, et al. The lack of effect of bupropion HCI (Wellbutrin) on the secretion of growth hormone and prolactin in humans. Life Sciences, 1982; 30(20):1725-32. In this case, dopamine transmission may not be critical to the clinical effect of bupropion.

The augmentation of bupropion to citalopram did not significantly change both transporter bindings which suggests that bupropion does not completely reverse the effects of citalopram to the baseline (without citalopram) level though it might affect the effects of citalopram. The change in the SERT (midbrain and diencephalon) by citalopram is consistent with its property and our expectation. The binding was decreased by approximately 40-50% according to the present finding.

The key observation that has led to this invention was the increase in striatal DAT binding by an SSRI medication. This is the first report of an SSRI directly effecting the population of striatal dopamine transporters, and this novel finding leads to an improved process for screening new pharmacologic candidates during drug discovery and development. The mechanism by which SSRI's exert this effect on the dopaminergic system is not well understood, however, it is likely related to serotonin and dopamine interaction. By increasing the DAT population, leading to increased uptake of more dopamine from synapse, the steady-state administration of a relatively “pure” SSRI (i.e., citalopram) causes a decrease in dopamine that is available at the synaptic cleft. Compared with the bupropion group that showed no significant change in the DAT binding, these findings explain the mechanism by which any SSRI is more likely to cause sexual dysfunction side effects than bupropion, since lowered dopaminergic tone is associated with this particular type of adverse effect.

It should be understood that the invention is not limited to the particular embodiments described herein, but that various changes and modifications may be made without departing from the spirit and scope of this novel concept as defined by the following claims.

What is claimed is:

1. A method for determining the activity of a drug affecting the central nervous system (CNS) on striatal dopamine transporter populations

2. A method of claim I wherein said CNS drugs are an antidepressant or anxiolitic agent.

3. The method of claim 2 wherein SPECT brain imaging, utilizing a I¹²³B-CIT radioligand, is used to predict the activity of antidepressant or anxiolytic medications on striatal dopamine transporter (DAT) populations.

4. The method of claim 3 wherein said SPECT scan is used to image the change in dopamine transporter (DAT) and serotonin transporter (SERT) populations caused by taking said antidepressant or anxiolytic agents.

5. The method of claim 4 wherein steady-state administration of said SSRI antidepressants or anxiolytic agents lead to an increase in said striatal DAT populations.

6. The method of claim 5 wherein the increase in striatal DAT is observed at steady-state dosing with a “pure” SSRI and is an SSRI class effect.

7. The method of claim 6 wherein the “pure” SSRI antidepressants or anxiolytic agents are selected from the group consisting of citalopram, fluoxetine hydrochloride, sertraline, fluvoxamine maleate, and paroxetine hydrochloride.

8. The method of claim 3 using the increase in striatal DAT population to predict the risk of sexual dysfunction side effects with said SSRI antidepressants or anxiolytic agents.

9. The method of claim 8 wherein a significant increase in the relative amount of radioligand binding to striatal DAT's as measured by an increased in the V3” ratio of between about 1.0 to about 2.0 units, predicts an increased risk of sexual dysfunction side effects.

10. The method of claim 9 using the increase in striatal DAT populations to determine the proper dosing for said SSRI antidepressants or anxiolytic agents to avoid said sexual dysfunction side effects.
